(19) United States
(12) Patent Application Publication
(10) Pub. No.: US 2016/0256513 A1
(43) Pub. Date: Sep. 8, 2016

(54) SYNERGIZED TURMERIC

(71) Applicants: Mitchell MAY, Castle Valley, UT (US); H.N. SHIVAPRASAD, Bangalore (IN)

(72) Inventors: Mitchell MAY, Castle Valley, UT (US); H.N. SHIVAPRASAD, Bangalore (IN)

(73) Assignee: The Synergy Company of Utah, LLC d/b/a Synergy Production Laboratories, Moab, UT (US)

(21) Appl. No.: 14/993,746

(22) Filed: Jan. 12, 2016

Related U.S. Application Data
(60) Provisional application No. 62/128,358, filed on Mar. 4, 2015.

(51) Int. Cl. A61K 36/9066; A61K 31/12; A61K 31/715

(52) U.S. Cl. CPC A61K 36/9066; A61K 31/715; A61K 31/12; A61K 2236/33; A61K 2236/51; A61K 2236/37

(57) ABSTRACT

A composition for enhanced bioavailability of curcumin, and methods of making and using as a health supplement. The composition includes a curcuminoid mixture, essential oil of turmeric, and water extract of turmeric polysaccharides. The composition is derived from turmeric rhizomes and provides bioavailability benefits in a subject in need thereof.

### CROSS REFERENCE TO RELATED APPLICATION

This application claims the benefit of U.S. Provisional Patent Application, Ser. No. 62/128,358, filed on 4 Mar. 2015. The co-pending Provisional Patent Application is hereby incorporated by reference herein in its entirety and is made a part hereof, including but not limited to those portions which specifically appear hereinafter.

### FIELD OF THE INVENTION

This invention relates generally to a turmeric supplement composition and, more particularly, to a composition for enhanced bioavailability of curcumin and a method of manufacture thereof that can provide a certified organic composition.

### BACKGROUND OF THE INVENTION

Curcumin is a major yellow pigment of turmeric (Curcuma longa), a well-known indigenous herbal medicine, and is a hydrophobic polyphenol derivative which is a potent antioxidant. Curcumin is a major active ingredient of turmeric, known for its diverse biological actions and pharmacological activities including anti-inflammatory, antioxidant, antiproliferative, antimicrobial, anticarcinogenic and antiangiogenic properties.

Commercial curcumin typically contains approximately: 77% diferuloylmethane, 17% demethoxycurcumin, and 6% bisdemethoxycurcumin. Curcumin 95% is the most common form of curcuminoid extract composition available in the market. This has a concentrate of 95% curcuminoids, but the bioavailability of the composition can be less than desirable. Compositions derived from dry turmeric have varying percentage of curcuminoid content, and without any excipients or formulation are generally poorly absorbed.

To improve the bioavailability of curcumin, numerous approaches have been undertaken. These approaches include the use of adjuvant like piperine that interferes with glucoronidation; the use of liposomal curcumin; the use of curcumin nanoparticles; the use of curcumin phospholipid complex; and the use of structural analogues of curcumin. Despite the lower bioavailability, preclinical studies have established the therapeutic efficacy of curcumin against various human diseases, including cancer, cardiovascular diseases, diabetes, arthritis, neurological diseases and Crohn's disease. Enhanced bioavailability of curcumin is likely to bring this promising natural product to the forefront of therapeutic agents for prevention and/or treatment of human disease.

US19985744121 observed that administering piperine along with curcumin enhances the bioavailability of curcumin. However the level of enhancement was only modest and no curcumin could be detected after 3 hours even when supplemented with piperine. Moreover, piperine has been shown to be toxic in animal studies.

US20070148263 relates to a formulation of curcuminoid with the essential oil of turmeric to enhance the bioavailability of curcumin and to augment the biological activity of curcumin. The disadvantage of this invention is that this formulation is also highly hydrophobic, insoluble, has strong turmeric oil aroma and acts by inhibiting glucuronidation, an important in vivo detoxifying mechanism.

WO2007103435 discloses a formulation comprising curcumin, an antioxidant, a water-solubilizing carrier, and optionally a glucoronidation inhibitor forming a curcuminoid-lipid micelle and finally provides as an emulsion or solid-lipid nanoparticles. The formula claims to have 5-times the bioavailability in animals upon 100 mg/kg level supplementation, as compared to standard curcumin. This formula, containing only 2 to 3% of curcumin, claims to have more than 5 times bioavailability upon 100 mg/kg level supplementation, Moreover, the clinical safety of nanoparticles for consumption has not been finalized.

WO2007101551 teaches phospholipid complexes of curcumin for improved bioavailability, and WO2010013224 describes the formation of chitosan bound curcumin nanoparticles with an enhancement of 10-fold bioavailability upon oral administration to rats. However, the process and recommended dilution during its preparation limits its commercial scale preparation.

In non-patent literature, Sasaki H et al, (Biol. Pharm. Bull., 2011, 34(5), 660-665) discloses colloidal nanoparticle suspension of curcumin in water comprising gum ghatti and glycerine as additives, prepared using a high pressure homogenization technique. Oral administration of the suspension was reported to enhance the bioavailability by 27 times. However, special equipment was required for its formulation along with chemical additives and non GRAS hydrocolloids. In addition, the reason for the enhanced bioavailability has been attributed to the nano form which is difficult to achieve in commercial scale. Additionally, the toxicity related to nanoparticles is not yet fully understood.

Curcumin extracts in the market, the common 95% curcuminoid extracts, and the variations for improved bioavailability, are all generally extracted with ethyl acetate, hexane, acetone, or combinations thereof These solvents pose significant health and safety concerns for the employees in the manufacturing facilities, environmental risks for the safe disposal of excess or used chemical solutions. Additionally, the presence of these solvents prevents the final composition from being certified organic.

There is a continuing need to develop a composition to enhance the bioavailability of curcumin to fully realize its potential.

### SUMMARY OF THE INVENTION

A general object of the invention is to improve the bioavailability of curcumin. The general object of the invention can be attained, at least in part, through a composition including curcumin and components that support enhanced bioavailability of the curcumin. The composition according to embodiments of this invention includes a curcuminoid mixture, essential oil of turmeric, and a water extract of turmeric polysaccharides. The presence of the essential oil of turmeric and the turmeric polysaccharides has been found to improve the bioavailability of curcuminoids. Further, the inclusion of curcuminoids, turmerones, and polysaccharides creates a composition that is inclusive of the naturally occurring compounds in turmeric, thereby yielding a whole spectrum extract. The inclusion of fat-soluble and water-soluble compounds improves the bioavailability of the composition. The invention also includes an oral pharmaceutical formulation including the composition of this invention, along with pharmaceutically acceptable additives. The invention further includes a method of providing antioxidant or other health benefits in a subject in need thereof, including the the steps of: administering an acceptable amount of the composition along with pharmaceutically acceptable additives, such as in a granule or tablet form, to the subject.

The invention further includes a method of preparing a turmeric composition, the method including: preparing a mixture of curcuminoids; extracting an essential oil of turmeric including turmerones; extracting turmeric polysaccharides; combining the mixture of curcuminoids, the essential oil of turmeric, and the turmeric polysaccharides to obtain the turmeric composition; and forming the turmeric composition into a granule. In preferred embodiments of this invention, the composition is produced using clean solvents, such as ethanol, water, and CO₂, whereby one or more of the solvents is desirably certified organic, and preferably the final composition may also be certified organic.

Other objects and advantages will be apparent to those skilled in the art from the following detailed description taken in conjunction with the appended claims and drawings.

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification are to be understood as being modified in all instances by the term “about.” It is noted that, unless otherwise stated, all percentages given in this specification and appended claims refer to percentages by dry weight of the total composition.

It is to be understood that this disclosure is not limited to particularly exemplified systems or process parameters that may of course, vary. It is also to be understood that the terminology used herein is for the purpose of describing particular embodiments of the invention only, and is not intended to limit the scope of the invention in any manner. Unless otherwise defined, all technical and scientific terms used herein have the same meaning as commonly understood by one of ordinary skill in the art to which this invention pertains. In the case of conflict, the present document, including definitions will control.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention. The terms “comprising,” “including,” “having,” “containing,” “involving,” and the like are to be understood to be open-ended, i.e., to mean including but not limited to.

As used herein, the term “curcuminoid” intends to mean natural polyphenols that are responsible for the yellow color of turmeric. “Turmeric” refers to Curcuma longa and any suitable breed, variation, or related species that would provide the components for the composition of this invention. Curcumin is a principal curcuminoid of turmeric rhizome. Curcumin can exist in tautomeric forms, including a 1,3-diketo form and two equivalent enol forms. Two other curcuminoids are demethoxycurcumin and bis-demethoxycurcumin.

### DETAILED DESCRIPTION OF THE INVENTION

The present invention provides a composition for enhanced bioavailability of curcumin, including a curcuminoid mixture, essential oil of turmeric, and a water extract of turmeric polysaccharides. The ratios of the components can vary, as discussed further below. In one embodiment of this invention, the ratio of the curcuminoid mixture to the added essential oil of turmeric and polysaccharides is about 80:10:10.

In embodiments of this invention, the curcuminoid mixture includes curcumin, demethoxycurcumin, and bisdemethoxycurcumin. The composition of one embodiment of this invention includes, by weight, from about 50% to about 85% curcuminoid mixture, more desirably about 60% to about 80%, and preferably about 65% to about 75%.

The essential oil of turmeric comprises turmerones, such as, without limitation, aromatic turmerones (Ar-turmerones), alpha turmerones (a-turmerones), beta turmerones (b-turmerones), or combinations thereof. The composition of embodiments of this invention includes, by weight, from about 2% to about 25% turmerones, more desirably about 2% to about 12%, and preferably about 2% to about 10%.

In embodiments of this invention, the composition includes carbohydrates extracted from turmeric. The carbohydrates are natural sugars present in the turmeric rhizomes, and the extracted carbohydrates are desirably mostly or fully polysaccharides. The polysaccharides are preferably water extracted from turmeric, such as from dried turmeric rhizomes. The composition of one embodiment of this invention includes, by weight, from about 1% to about 10% carbohydrates/polysaccharides, more desirably about 1% to about 5%, and preferably about 2%.

The composition of this invention is useful as a supplement to be taken by a human, such as for providing antioxidant or other health benefits. An acceptable amount of the composition is taken by or administered to a subject. The invention also includes an oral pharmaceutical formulation comprising the composition along with any one of more pharmaceutically acceptable additives. The composition can be formulated into dosage forms selected from tablets, capsules (e.g., vegetable capsules), troches, lozenges, aqueous or oily suspensions, dispersible powders or granules, emulsions in hard or soft gel capsules, syrups and elixirs, using additives selected from a group including, without limitation, granulating agents, binding agents, lubricating agents, disintegrating agents, sweetening agents, coloring agents, flavoring agents, coating agents, plasticizers, preservatives, suspending agents, and emulsifying agents. The amount (e.g., milligrams) of the composition can vary depending on need. In one embodiment of this invention, a capsule includes about 350 mg to about 650 mg of the composition.

The invention further includes a method of preparing the composition. In embodiments of this invention, the method includes preparing a mixture of curcuminoids, such as comprising curcumin, demethoxycurcumin, and bisdemethoxycurcumin, from turmeric, preparing essential oil with turmerones, preparing polysaccharides from turmeric, and combining the curcuminoids mixture, the essential oil of turmeric, and the water extract of polysaccharides to obtain the composition.

In one embodiment of this invention, the mixture of curcuminoids is extracted from turmeric in ethanol. The method can include distilling the ethanol to obtain a concentrated mixture, crystallizing the concentrated mixture to obtain crystals, treating the crystals with ethanol, and removing the ethanol by drying to obtain the mixture of curcuminoids from turmeric. The essential oil of turmeric extraction can be performed by fractional distillation of extracted turmeric components using ethanol. The essential oil of turmeric extraction can be additionally or alternatively performed by super critical CO₂ extraction from dried turmeric rhizomes.

The polysaccharides are preferably water extracted, such as from dried turmeric rhizomes. The extraction can be done in demineralized water, repeated as necessary. In one embodiment of this invention, the extracted turmeric polysaccharides are concentrated and then spray dried.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

A curcuminoid mixture was prepared by extracting certified organic turmeric with certified organic ethanol in a 1:2 ratio for three hours under continuous circulation at a temperature of about 40°-50° C. This process was repeated 7 more times and then the ethanol was distilled out at 60° C. in vacuum to get a concentrated certified organic oleoresin. Certified organic ethanol was added to the concentrated oleoresin for crystallization. Crystals obtained were again washed/treated with certified organic ethanol to get the final certified organic curcuminoid mixture, which was dried in vacuum tray drier at 80-90° C. for about 10 hours.

Certified organic essential oil of turmeric, containing more than 65% by weight of total turmerones, was prepared by fractional distillation using certified organic ethanol under series of temperatures: 140° C., 160° C., 180° C. and 200° C. Initially the oil was obtained in a certified organic ethanol extraction process of making a curcuminoids mixture and then purified by certified organic ethanol separation technique to remove the remaining curcuminoids content. The oil was purified to more than 65% total turmerones.

Additional essential oils of turmeric were obtained through a certified organic super critical CO₂ extraction of dried certified organic turmeric rhizomes. The oil was purified to more than 65% total turmerones.

A water extract of certified organic turmeric polysaccharides was prepared by extracting with DM water in 1:4 ratio for four times, followed by evaporating out the water at 90° C. in vacuum to get a concentrated liquid extract of 30% solids content, then spray dried to get certified organic turmeric water extract with more than 25% polysaccharides.

The composition was prepared by combining the above prepared curcuminoid mixture, essential oil of turmeric (one by ethanol extraction and one by super critical CO₂ extraction), and water extract of turmeric polysaccharides. The composition included 80% curcuminoid mixture, 10% essential oil of turmeric, and 10% water extract of turmeric polysaccharides. The composition was prepared by mixing, blending, and sieving the four components. The composition was dried in a vacuum tray drier and blended, then sieved to create one homogenous certified organic powder. The composition was granulated with 2% Certified Organic Acacia Gum to create free flowing granules.

As discussed above, studies on curcumin from turmeric indicate that it has relatively low bioavailability when administered orally. Previous studies revealed that approaches to enhance the bioavailability of curcumin include: 1) the use of piperine along with curcumin which interrupts the formation of curcumin conjugates; 2) micronization and the use of liposomal curcumin; 3) the use of nanotechnology; and/or 4) the use of soy lecithin and microcrystalline cellulose in curcumin formulation.

A bioavailable composition according to this invention, referred to as SYNR-1, and a commercially available curcuminoids 95% powder for comparison, were each assessed in human Caco-2 cell monolayers as an intestinal in vitro model system. The Caco-2 cell line exhibits a well-differentiated brush border on the apical surface with the tight junctions and has proved to be the most popular in vitro model. The Caco-2 cell model correlates with human intestine enterocytes and is used to predict oral absorption, and as a screening tool in drug discovery strategies for the prediction of intestinal drug permeability.

Test samples having different concentrations of permeable curcuminoids through Caco-2 cell monolayers were collected from a basolateral part in different time intervals. The percentage permeability and Pₐₚₚ values were calculated based on the ability of curcuminoids to cross the cell monolayers.

The percentage permeability of SYNR I was found to be 67.82, diffused across the Caco-2 cell monolayer. Apical-to-basolateral direction Apparent Permeability (Pₐₚₚ, in cm/sec) in Caco-2 cell monolayers of SYNR I was found to be 7.26×10⁻². The commercial 95% curcuminoids had a percentage permeability of 23.28 and Pₐₚₚ of 2.56×10⁻². The results indicated the potential increased bioavailability of the SYNR I formula of this invention as compared to commercially available 95% curcuminoids.

The turmeric oil standardized to turmerones having the lowest curcuminoids at a concentration of 0.213% showed quick absorption with percentage permeability of 93.21. The water extract standardized to polysaccharides tested to prove with 91.39 percentage permeability which had a concentration of 0.03% of curcuminoids. The presence of both these components worked efficiently in improving the bioavailability of SYNR I formula.

In order to study the bioavailability, SYNR I was applied to the apical side of a Caco-2 cell monolayer. The curcuminoids were quantified in the apical and basolateral sides after 4 h of incubation. The results obtained from the permeability studies, suggest that SYNR I had better bioavailability compared with 95% curcuminoids. Based on this when specified needs arise, developed formulation, ‘SYNR I’ can be given to maintain the continuous uphold of the curcuminoids in blood.

Thus, the invention provides a turmeric composition having improved bioavailability. The composition can be made with certified organic materials and methods, to provide a supplement with an antioxidant or other health benefit.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A composition for enhanced bioavailability of curcumin, comprising a curcuminoid mixture, essential oil of turmeric, and a water extract of turmeric polysaccharides.

2. The composition of claim 1, wherein the curcuminoid mixture comprises curcumin, demethoxycurcumin, and bisdemethoxycurcumin.

3. The composition of claim 1, wherein the curcuminoid mixture in the composition ranges by weight from about 65% to about 85%.

4. The composition of claim 1, wherein the essential oil of turmeric comprises turmerones.

5. The composition of claim 4, wherein the turmerones comprise aromatic turmerones (Ar-turmerones), alpha turmerones (a-turmerones), and beta turmerones (b-turmerones).

6. The composition of claim 1, wherein the essential oil of turmeric comprises turmerones in the composition range by weight from about 2% to about 12%.

7. The composition of claim 1, wherein the essential oil of turmeric comprises turmerones in the composition range by weight from about 2% to about 10%.

8. The composition of claim 1, wherein the polysaccharides in the composition range by weight from about 1% to about 10%.

9. The composition of claim 1, wherein ratio of the curcuminoid mixture to the added essential oil of turmeric and polysaccharides is about 80:10:10.

10. The composition of claim 1, wherein the composition is in a form of a granule.

11. The composition of claim 1, wherein the composition is certified organic.

12. An oral pharmaceutical formulation comprising the composition of claim 1 and pharmaceutically acceptable additives.

13. A method of preparing a turmeric composition, the method comprising:
extracting a mixture of curcuminoids;
extracting an essential oil of turmeric including turmerones;
extracting turmeric polysaccharides;
combining the mixture of curcuminoids, the essential oil of turmeric, and turmeric polysaccharides to obtain the turmeric composition; and
forming the turmeric composition into granules.

14. The method of claim 13, wherein the mixture of curcuminoids comprises curcumin, demethoxycurcumin, and bisdemethoxycurcumin, each obtained from turmeric.

15. The method of claim 13, further comprising extracting the mixture of curcuminoids from turmeric in ethanol.

16. The method of claim 15, wherein the ethanol is certified organic ethanol.

17. The method of claim 15, further comprising:
distilling the ethanol to obtain a concentrated mixture;
crystallizing the concentrated mixture to obtain crystals;
treating the crystals with ethanol; and
removing the ethanol by drying to obtain the mixture of curcuminoids from turmeric.

18. The method of claim 13, wherein the extracting the essential oil of turmeric comprises fractional distillation of extracted turmeric components using ethanol.

19. The method of claim 13, wherein the extracting the essential oil of turmeric comprises super critical CO₂ extraction from dried turmeric rhizomes.

20. The method of claim 13, further comprising:
extracting turmeric polysaccharides in demineralized water;
concentrating the extracted turmeric polysaccharides; and
spray drying the concentrated turmeric polysaccharides.
